(19) United States
(12) Patent Application Publication
(10) Pub. No.: US 2009/0204079 A1
(43) Pub. Date: Aug. 13, 2009

(54) CATHETERS WITH ENLARGED ARTERIAL LUMENS

(75) Inventors: Shekhar D. Nimkar, Swampscott, MA (US); Eric Tobin, North Andover, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: SPIRE CORPORATION, Bedford, MA (US)

(21) Appl. No.: 12/244,554

(22) Filed: Oct. 2, 2008

Related U.S. Application Data
(60) Provisional application No. 61/029,030, filed on Feb. 15, 2008.
(60) Provisional application No. 60/980,633, filed on Oct. 17, 2007.

(51) Int. Cl. A61M 25/14; A61M 25/00

(52) U.S. Cl. 604/246; 604/523

(57) ABSTRACT

Asymmetric lumen catheter devices are disclosed. In one aspect of the invention, a catheter assembly includes a first catheter tube having a first lumen extending longitudinally through the first catheter tube and a second catheter tube attached to the first catheter tube. The second catheter tube extends a longitudinal length beyond a distal end of the first catheter tube and has a second lumen extending longitudinally therethrough. The first lumen has a larger cross-sectional size than the second lumen.

### CROSS REFERENCE TO RELATED APPLICATIONS

The present application claims the priority of U.S. Provisional Application Ser. No. 60/980,633 filed Oct. 17, 2007 entitled “Manufacture of Split Tip Catheters” and U.S. Provisional Application Ser. No. 61/029,030 filed Feb. 15, 2008 entitled “Catheters With Enlarged Arterial Lumens,” which are herein incorporated by reference in their entireties. This application is also related to commonly owned U.S. patent application Ser. No. ______ filed concurrently herewith entitled “Fusion Manufacture of Multi-Lumen Catheters” (Attorney Docket No. 101430-236), and U.S. patent application Ser. No. ______ filed concurrently herewith entitled “Manufacture of Fixed Tip Catheters” (Attorney Docket No. 101430-240), each of which are herein incorporated by reference in their entireties.

### BACKGROUND

The present invention generally relates to catheters and preferably to multi-lumen catheters used for vascular access.

Multi-lumen catheters are desirable for various treatment applications such as hemodialysis where fluid extraction and return occur simultaneously. Hemodialysis is the separation of metabolic waste products and water from the blood by filtration. Typically, a hemodialysis unit is connected to a patient's body by a catheter. The catheter's distal end is placed in a blood vessel and its proximal end is connected to a hemodialysis unit.

During hemodialysis, a patient's blood typically flows through a double lumen catheter to the hemodialysis unit which provides filtration and controls the flow of blood. A double lumen catheter has two lumens that independently allow fluid extraction and return. For example, one lumen can be used for removing blood from a patient for processing in the hemodialysis machine and the other lumen can be used for subsequently returning the processed blood back to the patient's circulatory system. Such catheters can also include additional lumens for flushing, administration of anticoagulants or the like.

Parameters that can be varied to achieve adequate hemodialysis include blood flow rate, dialysis solution flow rate, and dialyzer competency. Generally, raising the blood flow rate increases dialysis efficiency. However, conditions such as access recirculation decrease efficiency. Access recirculation is the recirculation of treated blood back into the hemodialysis unit. Excess recirculation effectively reduces dialysis efficiency and lengthens the duration of the treatment needed for adequate dialysis. Access recirculation can be particularly of concern when using a double lumen catheter due to the close proximity of the intake and outflow ports at the distal tip of the catheter.

Various double lumen catheter designs have been suggested for the purpose of reducing access recirculation. For example, in so-called “staggered, fixed tip” designs, the distal ends of intake and outflow lumens can be longitudinally spaced 20-30 mm apart to prevent recirculation. For example, Twardowski et al. U.S. Pat. No. 5,569,182 discloses that the lumen for return of blood back into the vein should terminate beyond the extraction lumen. The purpose of this is to prevent cleansed blood, exiting from the outlet point of the catheter, from re-entering the catheter's blood inlet point and returning to the dialysis machine. However, certain disadvantages have been noted by such large longitudinal spacing between the distal ends of the respective lumens. For example, blood flow stagnation in the region of the blood vessel between two widely separated tips can lead to clot formation.

In addition to longitudinal spacing of the distal openings of the lumens, others have suggested that the distal end of a multi-lumen catheter can be split such that the distal tip segments can independently move in the blood vessel to optimize the fluid dynamics of the different functions (blood extraction and blood return). The introduction of an angle between the extraction and return lumens of a split tip catheter can further reduce the likelihood of access recirculation due to greater separation between inflow and outflow lumens.

Moreover, it can be desirable to have the maximum possible luminal cross-sectional areas to optimize catheter flow characteristics and also to maintain adequate flow over time since flow rates tend to decrease due to factors such as catheter clotting. However, there exists a need to maintain adequate physical and mechanical properties of the catheter, for instance tensile strength and kink-resistance, and to keep overall catheter dimensions small enough for insertion and proper physiological function. With these constraints in mind, it can be advantageous to have a different shape, e.g., greater luminal cross-section, for one or the other of the lumens or split tip segments, for example, to facilitate blood withdrawal or to diffuse returning cleansed blood. In particular, the arterial (or extraction) lumen is more prone to clogging and can benefit from having a larger cross-section. However, such geometric differences are difficult to incorporate into catheters using conventional manufacturing techniques.

While various catheters are known, there exists a need for more efficient and economical catheters, especially catheters where the distal openings of the lumens are longitudinally spaced or a different shape or geometry is desired for one or the other of the lumens or tip segments.

### SUMMARY OF THE INVENTION

Asymmetric lumen catheter devices are disclosed. In one embodiment of the invention, a catheter assembly includes a first elongate catheter tube having a substantially D-shaped cross-section over at least a portion of its length and having a first lumen extending longitudinally through the first catheter tube and a second elongate catheter tube adjacent to the first catheter tube. The second catheter tube extends a longitudinal length beyond a distal end of the first catheter tube, has a substantially D-shaped cross-section over at least a portion of its length, and has a second lumen extending longitudinally therethrough. The first lumen has a larger cross-sectional size than the second lumen over at least a portion of its length.

The catheter assembly can vary in any number of ways, such as by at least one of the tubes including a plurality of fluid passage holes. In some embodiments, at least one fluid passage hole can be formed in a side of a distal portion of at least one of the tubes, while in some embodiments, a hole can be formed at a distal end of at least one of the tubes.

The tubes can have a variety of configurations. For example, the tubes can be a unibody construction with the first and second lumens separated by a longitudinal septum. As another example, the tubes can be discrete elements where a longitudinal length of the first tube is attached to a portion of a longitudinal length of the second tube. The tubes can be fused along at least about 10%, preferably along at least about 50%, more preferably in some applications along at least about 70%, 80%, or 90% of the longitudinal lengths.

The distal and proximal portions of the catheter assembly can have any combination of split and fixed tips. For example, proximal portions of the tubes can be separate from each other.

The catheter assembly can include a variety of other features. For example, in some embodiments, the catheter assembly includes a lumen tip segment joined to a distal end of at least one of the tubes such that the lumen tip segment is in communication with the lumen of the tube to which it is joined. As another example, an outer sheath can encase the tubes along at least a portion of longitudinal lengths of the tubes. As yet another example, a flow diverting structure can be attached to an outside surface of a distal portion of the second catheter tube.

In another aspect of the invention, a catheter assembly includes two tubes, each tube having a lumen extending longitudinally therethrough and one tube having a larger luminal cross-sectional size than the other tube over at least a portion of their lengths. The tubes can be disposed adjacent to each other along at least a portion of respective longitudinal lengths of the tubes such that a distal portion of one tube extends longitudinally beyond a distal portion of the other tube. The catheter assembly also includes a flow diverting structure attached to the distal portion of the tube that extends longitudinally beyond the other tube.

The catheter assembly can vary in any number of ways, such as by at least one of the tubes including a plurality of fluid passage holes. In some embodiments, at least one fluid passage hole can be formed in a side of a distal portion of at least one of the tubes, while in some embodiments, a hole can be formed at a distal end of at least one of the tubes.

The tubes can have a variety of shapes, sizes, and configurations. For example, the tubes can be a unibody construction having a longitudinal septum extending therethrough. As another example, the tubes can be attached together along at least about 10%, preferably along at least about 50%, more preferably in some applications along at least about 70%, 80%, or 90% of the longitudinal lengths. In some embodiments, the tubes can each have at least one flat surface and be attached together along their flat surfaces. As another example, an outer sheath can encase the tubes along at least a portion of their longitudinal lengths.

The distal and proximal portions of the catheter assembly can have any combination of split and fixed tips. For example, proximal and/or distal portions of the tubes can be separate from each other.

The tubes can have any cross-sectional shape (e.g., substantially D-shaped, circular, etc.) and can have a cross-sectional shape the same as or different from the other tube along at least a portion of its longitudinal length.

The flow diverting structure can be attached to the distal portion of the tube (“the longer tube”) that extends longitudinally beyond the other tube (“the shorter tube”) in a variety of ways. For example, the diverting structure can be fused or glued to the longer tube.

The flow diverting structure can have a variety of shapes, sizes, and configurations. For example, the flow diverting structure can be oriented to intersect a longitudinal axis of the tube to which it is not attached. For another example, the flow diverting structure can have a diameter that does not exceed a diameter of the tube to which it is not attached. As another example, the flow diverting structure can be attached at a location between distal ends of the tubes.

The flow diverting structure can be composed of a variety of materials. For example, the diverting structure can be composed of a material different than a material of the tube to which it is attached, such as a material with a durometer that differs from that of the tube to which it is attached.

In another aspect of the invention, a catheter assembly includes first and second catheter tubes having first and second inner lumen extending longitudinally therethrough, respectively. The second catheter tube has a length greater than a length of the first catheter tube, and the first inner lumen has a cross-sectional size that is larger than a cross-sectional size of the second inner lumen. A distal portion of the first inner lumen has a cross-sectional size that is different from the cross-sectional size in its non-distal portion.

The tubes can have a variety of configurations. For example, the tubes can be a unibody construction with the first and second lumens separated by a longitudinal septum. As another example, the tubes can be discrete elements where a longitudinal length of the first catheter tube is attached to a portion of a longitudinal length of the second catheter tube.

The tubes can have any cross-sectional shape (e.g., substantially D-shaped, circular, etc.). The cross-sectional size of the distal portion of the first inner lumen can be larger than the cross-sectional size in its non-distal portion.

The inner lumens can have any cross-sectional shape (e.g., substantially D-shaped, circular, etc.). In some embodiments, one of the first and second inner lumens can have a cross-sectional shape different from the other inner lumen along at least a portion of its longitudinal length.

The catheter assembly can vary in any number of ways, such as by at least one of the tubes including a plurality of fluid passage holes. In some embodiments, at least one fluid passage hole can be formed in a side of a distal portion of at least one of the tubes, while in some embodiments, a hole can be formed at a distal end of at least one of the tubes.

The distal and proximal portions of the catheter assembly can have any combination of split and fixed tips. For example, proximal and/or distal portions of the tubes can be separate from each other.

The catheter assembly can include a variety of other features. For example, in some embodiments, the catheter assembly includes a lumen tip segment joined to a distal end of the first catheter tube such that the lumen tip segment is in communication with the first inner lumen. As another example, an outer sheath can encase the tubes along at least a portion of longitudinal lengths of the tubes. As yet another example, a flow diverting structure can be attached to a distal portion of the second catheter tube.

Other advantages and features will become apparent from the following description and from the claims.

### BRIEF DESCRIPTION OF THE DRAWINGS

The invention will be more fully understood from the following detailed description taken in conjunction with the accompanying drawings, in which like reference numerals designate like parts throughout the figures, and wherein:

FIG. 1 is a schematic view of an embodiment of the present invention showing a multi-lumen catheter;

FIG. 2 is a schematic view of another embodiment of the present invention showing a multi-lumen catheter having a split tip proximal end;

FIG. 3 is a schematic view of an embodiment of the present invention showing a multi-lumen catheter having an angled end portion;

FIG. 4 is a schematic view of an embodiment of the present invention showing a multi-lumen catheter with separable tip portions held together by an adhesive;

FIG. 5 is a schematic view of an embodiment of the present invention showing a multi-lumen catheter including differently shaped lumens;

FIG. 6 is a cross-section view of an embodiment of the present invention showing a catheter construction utilizing opposed D-shaped lumens of different cross-sectional areas;

FIG. 7 is a cross-section view of an embodiment of the present invention showing a catheter construction with two individual circular lumens of different cross-sectional areas;

FIG. 8 is a cross-section view of an embodiment of the present invention showing an oval-shaped catheter construction with two individual circular lumens of different cross-sectional areas;

FIG. 9 is a schematic, partially cutaway, side view of a catheter according to the present invention;

FIG. 10 is a cross-section view of an embodiment of the present invention showing a catheter construction formed from opposed D-shaped lumen bodies inside an outer sheath;

FIG. 11 is a cross-section view of an embodiment of the present invention showing a catheter construction formed from two individual tubes with circular lumens inside an outer sheath;

FIG. 12 is a schematic, perspective view of a catheter according to the present invention in an initial, pre-trimmed configuration;

FIG. 13 is a schematic, perspective view of another catheter in an initial, pre-trimmed configuration;

FIG. 14A is a schematic, perspective view of an embodiment of the present invention showing a trimmed catheter;

FIG. 14B is a schematic, perspective view of a variation of an embodiment of the present invention showing a trimmed catheter;

FIG. 15A is a cross-sectional view of another multi-lumen catheter construction according to the invention;

FIG. 15B is another cross-sectional view of the catheter construction of FIG. 15A following partial removal of the larger luminal tube;

FIG. 15C is a cross-sectional view of the catheter construction of FIG. 15B following attachment of a new single-D tube;

FIG. 15D is a cross-sectional view of the catheter construction of FIG. 15C illustrating differences in cross-sectional profile between the original tube segment that is removed and its replacement;

FIG. 16 is a schematic, perspective view of an embodiment of the present invention showing a lumen tube attached to a catheter;

FIG. 17 is a distal cross-sectional view of another embodiment of the present invention showing alternative adhesive disposition;

FIG. 18 is a distal cross-sectional view of yet another adhesive design;

FIG. 19 is a schematic, perspective view of a variation of an embodiment of the present invention showing a lumen tube attached to a catheter;

FIG. 20 is a schematic, perspective view of a variation of an embodiment of the present invention showing a lumen tube attached to a catheter;

FIG. 21 is a schematic, perspective view of a variation of an embodiment of the present invention showing a lumen tube attached to a catheter, where the lumen tube is attached to at least a portion of the septum;

FIG. 22 is a schematic, perspective view of a variation of an embodiment of the present invention showing a lumen tube attached to a catheter, where the lumen tube is attached to at least a portion of the septum using an alternative method;

FIG. 23 is a schematic, perspective view of an embodiment of the present invention showing fluid openings in the distal tip;

FIG. 24 is a schematic view of an embodiment of the present invention showing a multi-lumen catheter having a flow diverting structure attached thereto;

FIG. 25 is a partial cutaway, side view of another embodiment of the present invention showing a multi-lumen catheter having a flow diverting structure attached thereto;

FIG. 26 is a top view of the multi-lumen catheter of FIG. 25;

FIG. 27 is a cross-section view of an embodiment of the present invention showing a catheter construction utilizing a D-shaped lumen and a D-shaped flow diverting structure;

FIG. 28 is a cross-section view of an embodiment of the present invention showing a catheter construction with a circular lumen and a D-shaped flow diverting structure;

FIG. 29 is a cross-section view of an embodiment of the present invention showing a variation of a catheter construction with a circular lumen and a D-shaped flow diverting structure;

FIG. 30 is a cross-section view of an embodiment of the present invention showing a catheter construction utilizing two D-shaped lumens and a D-shaped flow diverting structure; and

FIG. 31 is a cross-section view of an embodiment of the present invention showing a catheter construction with a circular lumen and an arced flow diverting structure.

### DETAILED DESCRIPTION

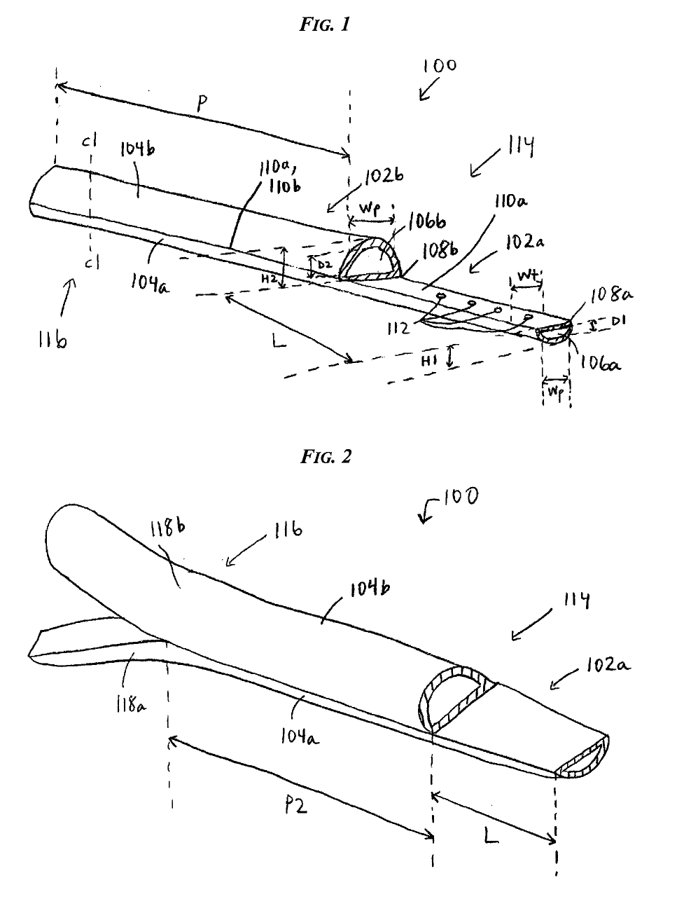

In FIG. 1 an embodiment of a catheter assembly 100 according to the invention is shown having first and second catheter tubes or bodies 104a, 104b (collectively, the tubes or bodies 104). (As used throughout, “the catheter assembly” and its components refers to the various embodiments of the present invention.) The tubes 104 include respective first and second inner lumen pathways 106a, 106b (collectively, the pathways 106) extending longitudinally through the tubes 104 for, e.g., the extraction or return of blood or other bodily fluids. The entire longitudinal length of the second tube 104b (also referred to as “the shorter tube 104b”) is attached to the first tube 104a (also referred to as “the longer tube 104a”), leaving a freely floating, unattached distal tip portion 102a of the longer tube 104a having a distal end 108a that extends a longitudinal length L beyond a distal end 108b of the second tube 104b (also referred to as “the shorter tube 104b”). The length L can be in the range of about 0.5-3 inches, which is a preferable, but only an example, length of the distal tip portion 102a. The distal ends 108a, 108b (collectively, the distal ends 108) of the tubes 104 can be open to provide fluid passageways through the pathways 106, e.g., for blood removal and return. Each of the tubes 104 in this illustrated embodiment has a substantially D-shaped cross-section and at least one substantially flat surface (e.g., facing or contacting surfaces 110a, 110b (collectively, the facing or contacting surfaces 110)). The tubes 104 can, however, have different cross-sectional shapes.

The first pathway 106a is of a smaller size (e.g., smaller cross-sectional area) than the second pathway 106b. Either of the pathways 106 can have a larger cross-sectional area than the other pathway, but the larger pathway is typically in the shorter, arterial tube 104b because that is the one of the tubes 104 more prone to clogging in a hemodialysis setting, and a larger size pathway 106b can help reduce clogging. The pathways 106 can have different diameters or heights, such as in the illustrated embodiment where a diameter D2 of the second pathway 106b exceeds a diameter D1 of the first pathway 106a. The tubes 104 can also have different sizes (e.g., cross-sectional areas). Either of the tubes 104 can have a larger size, but in this embodiment, a diameter or height H2 of the second tube 104b exceeds a height H1 of the first tube 104b. Although the tubes 104 and the pathways 106 are shown having equal widths Wt and Wp, respectively, the tubes 104 and/or the pathways 106 can have different widths. In some embodiments, the pathways 106 can have the same diameter but still have different cross-sectional areas, e.g., by varying one or both of the tube and pathway widths Wt, Wp.

The catheter assembly 100 has fixed tip distal and proximal portions 114, 116, although the catheter assembly 100 can have any combination of fixed tips and split tips at its distal and proximal portions 114, 116. An outer sheath can be added to at least a portion of the catheter assembly 100, as discussed further below, and/or access ports can be added to the tubes 104 at the proximal portion 116. The access ports can include couplings, such as Luer-locks or the like, to couple the proximal portion 116 to a hemodialysis machine in which blood is circulated and purified. The catheter assembly 100 is typically a very flexible silicone, polyurethane, or other biocompatible composition (e.g., having a stiffness in the range of about 65 to about 85 durometer), and can be fabricated into any type of catheter (e.g., a hemodialysis catheter or a central venous catheter).

The catheter assembly 100 can be formed in a variety of ways. In some embodiments, the catheter assembly 100 can be formed by trimming one of the tubes 104 to a longitudinal length less than a longitudinal length of the other tube. In other embodiments, the tubes 104 can be attached together to form the catheter assembly 100. For example, in one embodiment, the tubes 104 can be fused along at least a portion of their longitudinal lengths along substantially flat surfaces, such as the contacting surfaces 110 of the tubes 104. Any fusion technique can be used, e.g., thermal fusion where elements to be joined (here, outer surfaces of the tubes 104) are heated along any or all portions of their perimeters or other areas to a desired temperature and fused together by application of a desired force and allowing them to melt/cool together. In another example embodiment, the tubes 104 can be fused together using a bonding technique, e.g., applying a bonding material such as an adhesive to one or more of the elements to be bonded and, if necessary, heating the bonding material to bond it to the elements. In some embodiments, the catheter assembly 100 can be formed using any combination of heat fusion and bonding techniques. Whether or not the tubes 104 have equal longitudinal lengths, the catheter assembly 100 can be formed by extending the tubes 104 in a staggered, step configuration such that one of the tubes 104 extends longer than the other tube at the distal portion 114 and/or the proximal portion 116 by any length. By non-limiting example, the tubes 104 can be aligned while hot so at least one of the tubes 104 longitudinally extends beyond the other at the distal and/or proximal portions 114, 116 and can bond together in such a formation as they cool.

In some embodiments, a lumen tip segment of any length can be joined to one of the tubes 104 such that the distal portion 114 of that tube 104 includes the lumen tip segment, such that the lumen tip segment is in fluid communication with the pathway 106 of the tube to which the lumen tip segment is attached, and such that the longer tube 104a extends the length L beyond the distal end 108b of the shorter tube 104b. Prior to joining the lumen tip segment to one of the tubes 104, that tube 104 can be trimmed, as discussed further below.

Any portion of each of the tubes 104 can be attached together, e.g., 100% of the longitudinal lengths of one or both tubes 104, about 90% of the longitudinal lengths of one or both tubes 104, etc. If less than 100% of the tubes' longitudinal lengths are attached together, the resulting catheter assembly 100 can be used to create a split tip catheter, e.g., by adding one or more additional structures to the catheter assembly 100. As illustrated in FIG. 1, the tubes 104 are attached together along a portion P of their lengths, including the entire length P of the shorter tube 104b and leaving the longer tube's freely floating, unattached distal tip portion 102a of length L. In another embodiment, shown in FIG. 2, the tubes 104 can be attached together along a portion P2 of their longitudinal lengths, leaving the longer tube's distal tip portion 102a of length L at the distal portion 114 and leaving freely floating, unattached portions (lumen tip segments 118a, 118b (collectively, the lumen tips 118)) at the proximal portion 116.

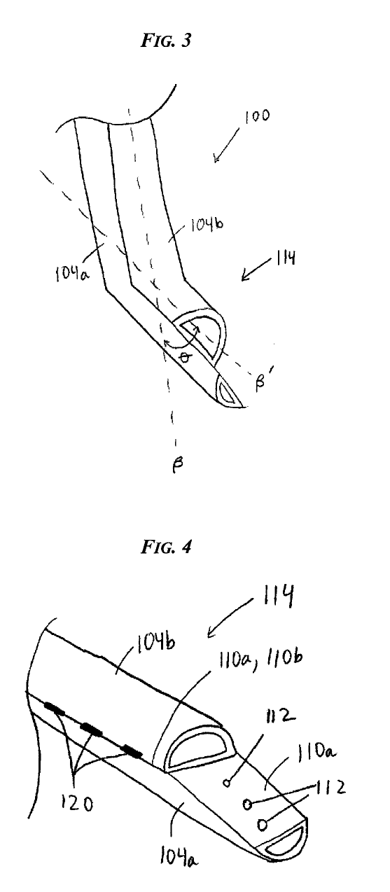

The catheter assembly embodiments illustrated in FIGS. 1-2 show the tubes 104 linearly aligned and substantially parallel to each other along their longitudinal lengths. However, as shown in FIG. 3, the tubes 104 at the distal portion 114 (and/or at the proximal portion 116 (not shown in FIG. 3)) can be substantially parallel to each other in an angled tip configuration, e.g., as described in U.S. Pat. No. 6,482,169, which is hereby incorporated by reference in its entirety. In such a configuration, the distal portion 114, having a distal longitudinal axis β′, is oriented at an angle θ with respect to a longitudinal axis β of the non-angled portion of the catheter assembly 100, where θ can have any value (including zero). The angle θ can be formed after the tubes 104 have been joined, e.g., by the application of heat. Alternatively, the tubes 104 can have an initial configuration where the distal axis β′ is at the angle θ with respect to the axis β.

Also illustrated in FIG. 1 are fluid passage holes (also called fluid openings) 112. The distal tip portion 102a of the longer tube 104a can, but need not, have one or more fluid passage holes 112 in fluid communication with its inner pathway 106a to facilitate fluid removal or return as appropriate, e.g., blood removal or return during hemodialysis. The fluid openings 112 can be of any number, shape, and size and can be located in a variety of places on any of the tubes 104. The fluid openings 112 can be formed in one or more of the tubes 104 prior and/or subsequent to joining the tubes 104 (if the catheter assembly 100 is formed by joining the tubes 104, as opposed to a unibody construction where the pathways are separated by a longitudinal septum). FIG. 3 shows the fluid openings 112 located on the facing surface 110a of the longer tube 104a. Alternatively, or in conjunction with the fluid passage holes 112, one or both of the distal ends 108 of the tubes 104 can be open to provide fluid passageways through the pathways 106. A distal tip portion 102b of the shorter tube 104b can similarly have, but need not have, one or more fluid passage holes in fluid communication with its inner pathway 106b. Fluid openings in the shorter tube 104b could be exposed, for example, by not fusing the distal portion 114 of the shorter tube 104a to the longer tube 104b or by allowing one or more fluid openings to be exposed upon dissolution of bioresorbable adhesive filling or covering the fluid openings, as described further below.

In an embodiment shown in FIG. 4, bioresorbable adhesive can be applied to at least a portion of the facing surfaces 110 of the tubes 104 as discrete spots or regions 120. As used herein, the term “bioresorbable” refers to materials that are biodegradable or biosoluble such that they degrade or break down by mechanical degradation upon interaction with a physiological environment into components that are metabolizable or excretable over a period of time.

The bioresorbable adhesive used to join the tubes 104 to one another can be a composition selected from the group of polymers consisting of polylactides, polyglycolides, polylactones, polyorthoesters, polyanhydrides, and copolymers and combinations thereof. In general, bioresorbable adhesives have bonding elements and degradable elements. The degradable elements can have the components of polylactide, polyglycolide and polylactones (polycaprolactone). The bonding elements can have hydrogen bonding strength (polyvinyl alcohol, polysaccharides) or can be able to polymerize as a single component (cyanoacrylates) or as two components (epoxy compound plus amino compounds, or radical (light) initiators of acrylate compounds).

Proteins, sugars, and starch can also be used as an adhesive. By way of non-limiting example, antithrombotic agents such as heparin and hirudin, citrate, antithrombin-heparin complex, and albumin heparin complex as well as anti-infective agents such as chlorohexidine, silver, antibiotics, and antiseptic agents may be added to the adhesive.

In an embodiment of the present invention, polymers which can be useful include polyurethane, generally described as a copolymer of polyethylene glycol with polylactide or polyglycolide end capped with methacrylates. Another embodiment can include a two component composition, one component preferably including a low molecular weight polyurethane end capped with methacrylates, and the other component preferably including polylactide, polyglycolide, or polycaprolactone end capped with methacrylate.

In another embodiment of the present invention, one or more components can be used from styrene, methyl methacrylate, methyl acrylate, ethylene dimethacrylate, ethylene diacrylate, acrylamide, diurethane dimethacrylate, polyisoprenegraft-maleic acid monomethyl ester, azobis (cyanovaleric acid), azobiscyclohexanecarbonitrile, azobisisobutyronitrile, benzoyl peroxide, iron (II) sulfate, polyvinyl alcohol, dextran, polysaccharide, epichlorohydrin, ethylenediamine, diaminocyclohexane, diamino propane, copolymers with polylactide and polyethylene oxide as the blocks and acrylate, methacrylate as the end groups, cyanoacrylates, ethyl-2cyanoacrylate, propyl-2-cyanoacrylates, pentyl-2-cyanoacrylate, hexyl-2-cyanoacrylate, and octyl-2-cyanoacrylate, ammonium persulfate and/or polyethylene glycol methacrylate when water, organic solvent such as dichloromethane, chloroform, tetrahydrofuran, acetone, petroleum ether, acetyl acetate, dimethylformamide, or the mixture thereof, is combined with the aforementioned solvents.

Additional information on bioresorbable adhesive compositions and catheter assembly manufacturing techniques employing such compositions can be found in commonly-owned, co-pending U.S. patent application Ser. No. 10/874,298 filed Jun. 9, 2004 entitled “Splitable Tip Catheter With Bioresorbable Adhesive”, herein incorporated by reference in it entirety.

The spots 120 of the bioresorbable adhesive can be applied continuously along the entire longitudinal length of the tubes 104 or selectively in an assortment of areas thereof. Preferably, the bioresorbable adhesive is applied such that the spots 120 of adhesive facilitate the joining of the distal portions 114 of the tubes 104 prior to insertion into a blood vessel and allow the distal portion 114 of the tubes 104 to separate after insertion. The spots 120 of bioresorbable adhesive can vary in number, size, and distance from one another in order to facilitate the joining and/or disjoining of the tubes 104. Preferably, the bioresorbable adhesive is applied along non-fused portions of both of the facing surfaces 110 such that the spots 120 of adhesive facilitate the joining of the tubes 104 prior to insertion into a blood vessel and allow the distal portions 114 of the tubes 104 to separate after insertion. Also preferably, if a lumen tip segment is attached to one of the tubes 104, the bioresorbable adhesive is applied prior to attachment of the lumen tip segment.

In the embodiments described herein, the bioresorbable adhesive preferably dissolves after insertion into a blood vessel to provide separation of the tubes 104 in a time period, e.g., over a period of time ranging from 1 second to several days (or longer), more preferably from about one minute to about ten hours, or five hours or one hour. This time period can be controlled by using different compositions of the bioresorbable adhesive as well as by the amount of adhesive applied to join the tubes 104 together. In an embodiment of the catheter assembly 100 with one or more distal fluid openings 112, the bioresorbable adhesive can be water soluble such that the introduction of saline or similar type fluid will effectuate the separation of the tubes 104 and exposure of one or more of the fluid openings 112. In this instance, the bioresorbable adhesive will not dissolve until a time after the introduction of the soluble solution into the tubes 104. Furthermore, the fluid openings 112 can be filled or covered with fluid activated bioresorbable adhesive, whether or not bioresorbable adhesive is otherwise used on the facing surfaces 110 of the tubes 104. After insertion of the catheter assembly 100 into a blood vessel, saline or similar type fluid can be introduced into one or both of the tubes 104 at the open proximal portion 116 such that the fluid travels through the tube(s) 104 to the distal fluid openings 112 and dissolves the fluid activated bioresorbable adhesive, thereby allowing fluid communication between the openings 112 and the lumen pathway(s) 106. The openings 112 are obscured on the shorter tube 104b until such time one or more of the spots 120 of adhesive dissolve and provide fluid access to one or more of the openings 112. Of course, depending on the lengths of the tubes 104, one or more openings 112 on both of the tubes 104 could be obscured until such time one or more of the spots 120 dissolve and/or adhesive filling or covering the openings 112 dissolves.

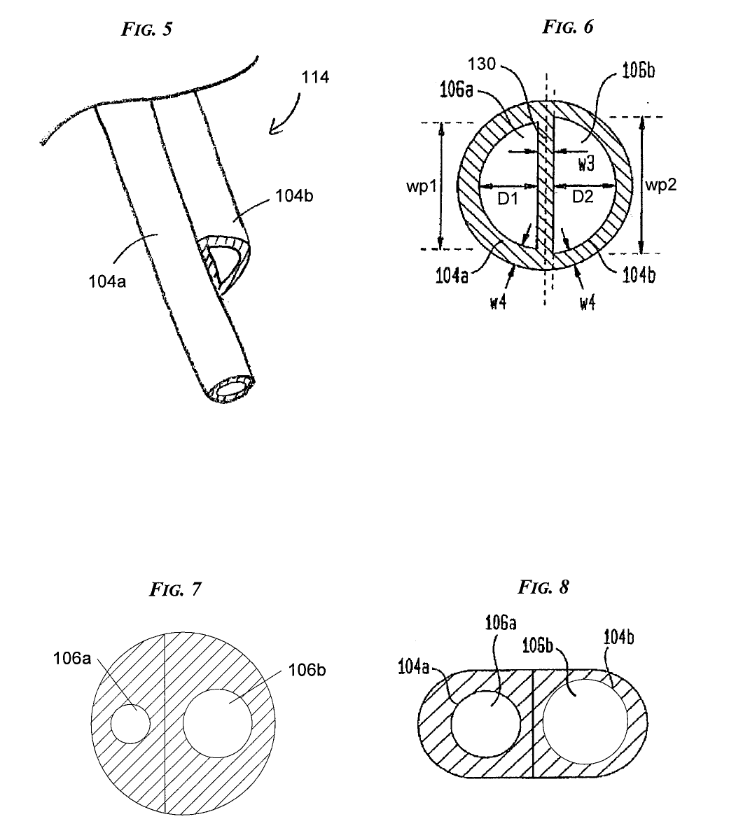

The tubes 104 can have a variety of cross-sectional shapes and sizes but preferably, as shown in the embodiments of FIGS. 1-4, the catheter assembly 100 has a substantially elliptical (circular or oval) shape and the tubes 104 are each substantially D-shaped. However, one or both of the tubes 104 can transition from one shape to another along at least a portion of its length, e.g., transition from a D-shaped cross-section to a circular cross-section. Furthermore, each of the tubes 104 can have a cross-sectional shape or size that can be the same or distinct from the catheter assembly 100 and/or the other tube. One embodiment of the catheter assembly 100 where the tubes 104 have different cross-sectional shapes at least in the distal portion 114 is shown in FIG. 5, with one tube 104b and pathway 106b having D-shaped cross-sections and the other tube 104a and pathway 106a having substantially circular cross-sections. A substantially flat-sided surface of the D-shaped tube 104a can be attached to a substantially flat, tangential surface of the substantially circular tube 104b.

Examples of c1-c1 cross-sections (see FIG. 1) of the catheter assembly 100 are illustrated in FIGS. 6-8. FIG. 6 is a c1-c1 cross-section view of an embodiment showing a construction utilizing opposed D-shaped tubes 104 where one tube 104a is of a smaller size (e.g., smaller cross-sectional area) than the other tube 104b. Dimensions of catheter assembly 100 can vary between embodiments. In this example embodiment, dimensions allow the catheter assembly 100 to be used with standard hemodialysis equipment and lumen tip segments. Maximum diameter D1 of the smaller pathway 106a is about 0.06 in. and maximum diameter D2 of the larger pathway 106b is about 0.08 in. A septum 130 has a width w3 of about 0.02±0.002 in., while the tubes 104 have an exterior width w4 of about 0.022±0.003 in. Maximum height wp1 of the smaller pathway 106a is about 0.14 in. and maximum height wp2 of the larger pathway 106b is about 0.15 in. FIG. 7 is a c1-c1 cross-section view of an embodiment showing an elliptical construction utilizing individual, elliptical lumen pathways 106. FIG. 8 is a c1-c1 cross-section view of another embodiment showing another elliptical construction including D-shaped tubes 104 and two elliptical-shaped pathways 106 in the tubes 104.

As mentioned above, an outer sheath, e.g., a fusing tube, can be added to partially or entirely cover and enclose the catheter assembly 100. Such an outer sheath can encase the catheter assembly 100 and smoothen any irregularities along at least part of the attached portion of the longitudinal lengths of the tubes 104. The outer sheath can be any shape and size and can be made of the same material as the tubes 104 or other material compatible with insertion into a blood vessel. The outer sheath can remain on or be removed from at least a portion of the catheter assembly 100. FIG. 9 illustrates an embodiment of the catheter assembly 100 partially encased by an outer sheath 122 and formed into a split tip catheter 124. As illustrated in this embodiment, the outer sheath 122 terminates proximal to the distal ends 108 of the tubes 104 such that the distal tip portion 102a of the longer tube 104a is separate from the shorter tube 104b. Also shown in FIG. 9 is the proximal portion 116 of the catheter assembly 100 split into the separate lumen tips 118 that terminate with two access ports 126a, 126b.

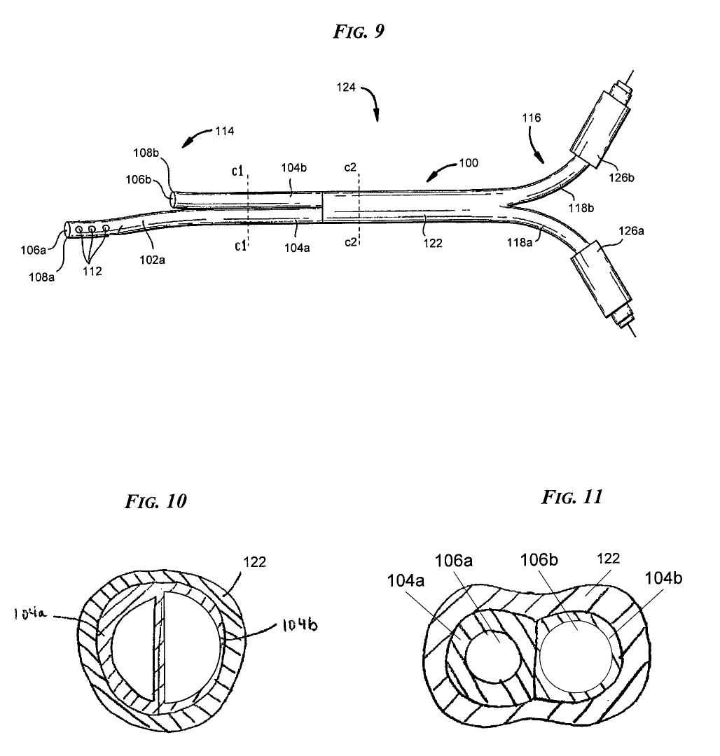

FIG. 10 shows a cross-section c2-c2 (see FIG. 9) of one embodiment of the outer sheath 122. The outer sheath 122 can be of any thickness and can have varying inner and outer shapes as well as varying inner and outer dimensions. The catheter assembly 100 can be constructed such that sheath material encases the tubes 104 and no space remains between the sheath 122 and the tubes 104. For example, the sheath 122 can be fused to the tubes 104 or heat-shrunk around them. FIG. 11 shows another embodiment of the cross-section c2-c2 showing individual, elliptical tubes 104 having substantially circular cross-sectional pathways 106 inside the outer sheath 122.

As mentioned above, the catheter assembly 100 can be formed by attaching the tubes 104 together. Additional information on such catheter assemblies and catheter assembly manufacturing techniques can be found in commonly-owned, co-pending U.S. patent application Ser. No. ______ filed concurrently herewith entitled “Fusion Manufacture of Multi-Lumen Catheters” (Attorney Docket No. 101430-236).

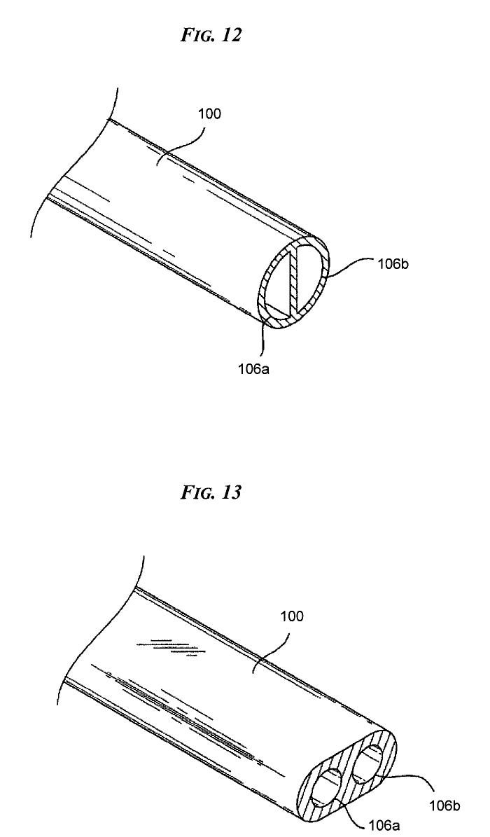

Also as mentioned above, in some embodiments, the catheter assembly 100 can be formed by trimming one of the tubes 104 such that at the distal end 114 one of the tubes 104b is shorter than the other one of the tubes 104a. Additional information on such catheter assemblies and catheter assembly manufacturing techniques can be found in commonly-owned, co-pending U.S. Provisional Application Ser. No. 60/980,633 filed Oct. 17, 2007 entitled “Manufacture of Split Tip Catheters.” An exemplary method of forming a catheter is described with reference to FIGS. 12-23. Although described with reference to these figures (and related ones of FIGS. 1-11), this method (or a similar method) can be implemented to form any of the catheter devices described herein.

FIG. 12 shows a circular catheter assembly 100 in an initial, untrimmed configuration (e.g., without separate distal tip segments) having two D-shaped pathways 106a, 106b. FIG. 13 shows another, elliptical catheter assembly 100 with circular pathways 106a, 106b in an initial, configuration (e.g., prior to trimming and, optionally, joinder of a distal lumen tip segment). Although the tubes 104 are shown having equal lengths at the distal end 114 in FIGS. 12-13, the tubes 104 can have different lengths in this initial configuration.

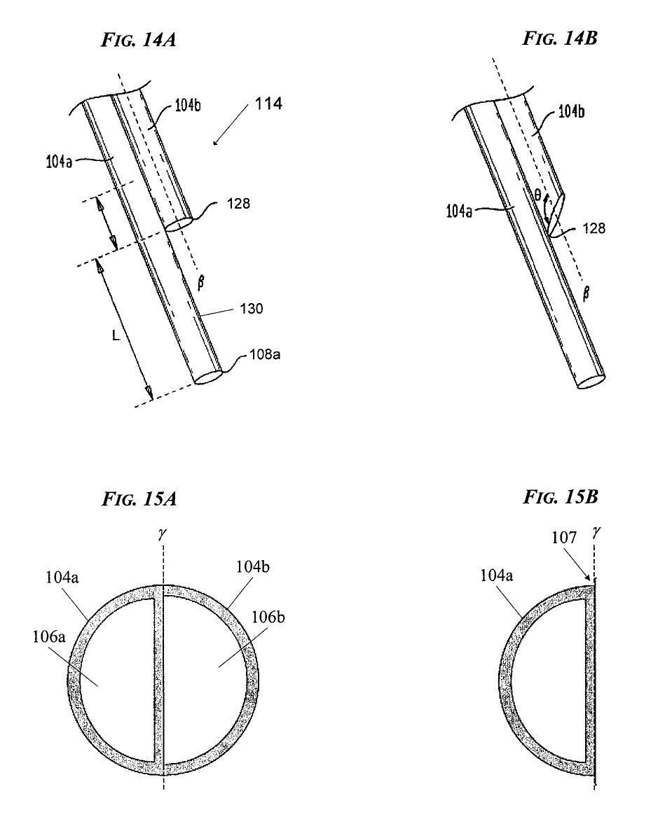

FIG. 14A shows the catheter assembly 100 in a trimmed configuration where a distal portion of the catheter body 100 has been removed, as compared to the initial configuration in FIG. 12 or 13. The catheter assembly 100 of FIG. 14A can also be formed by extending the tubes 104 in a staggered, step configuration such that one of the tubes 104a is extended longer than the other tube 104b by the length L. However formed, in this configuration, the longer tube 104a (also referred to as “the uncut tube 104a”) extends longitudinally beyond the shorter tube 104b (also referred to as “the cut tube 104b”) by the length L. In an initial configuration such as in this embodiment where the tubes 104 initially have equal lengths in the distal portion 114, the length L equals the amount of tube trimmed from the cut tube 104b.

The cut tube 104b can be trimmed in a variety of ways. In a preferred example, one of the tubes 104b can be sliced (e.g., cut or scored) widthwise across its circumference at a location 128. Then the length L of the cut tube 104b can be trimmed from the catheter assembly 100. When the length L of the cut tube 104b has been removed, a septum 130 between the cut tube 104b and the uncut tube 104a can thereby be at least partially exposed.

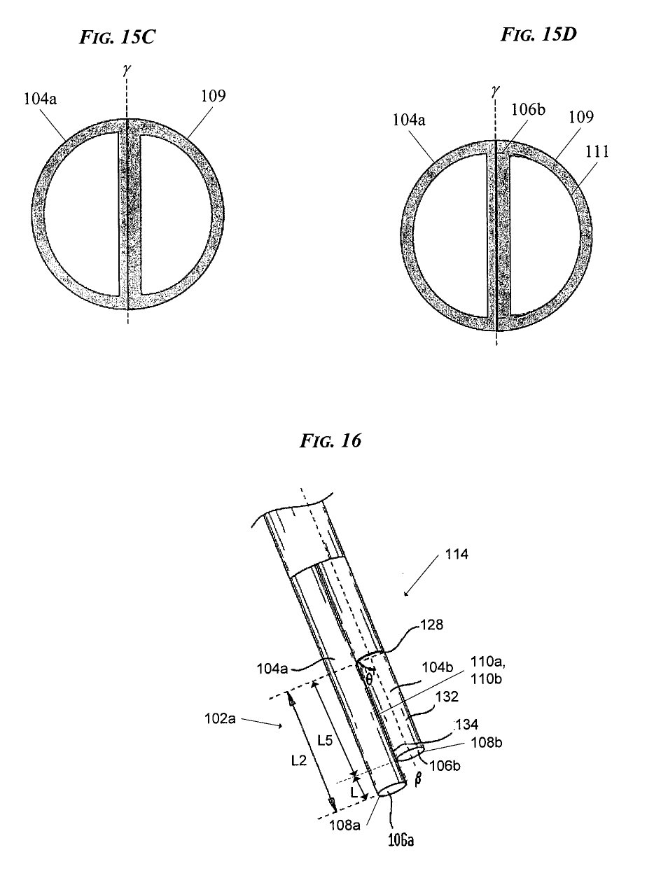

Truncation of the end portion according the invention typically involves sacrificing part of the tube 104b having a larger cross-sectional area and joining a new distal tip segment in its place. As illustrated for example in FIG. 15A, the catheter assembly 100 can be split along a longitudinal axis γ. The longitudinal axis γ corresponds to a bottom (flat base) of the tube 104b having a larger cross-sectional area and also to a centerline of the catheter assembly 100. As shown in FIG. 15B, a portion of the shorter tube 104b having a larger pathway 106b can be removed along the longitudinal axis γ down to a septum 107. A lumen tip segment, e.g., a new single-D tube 109, can be attached to the septum 107, as shown in FIG. 15C. The new lumen tip segment 109 has a slightly smaller cross-sectional area than the lumen 106a of the longer tube 104a. Looking at the catheter assembly 100 end-on from its distal end 114, as shown in FIG. 15D, the new lumen tip segment's pathway 111 has a smaller cross-sectional area than the original tube's pathway 106b, which is still in the background from the point where the new lumen tip segment 109 attaches through the catheter assembly's proximal end 116.

In certain applications it can be preferable to sacrifice the smaller tube 104a instead. In such instances, the truncation line can be moved to the other side of the septum 107.

The cut distal end 128 of the cut tube 104b can be trimmed in a perpendicular direction or a non-perpendicular direction with respect to a longitudinal axis β of the cut tube 104b. FIG. 14A shows the cut distal end 128 trimmed in a perpendicular direction with respect to axis β. Alternatively, FIG. 14B shows the cut distal end 128 trimmed in a non-perpendicular direction with respect to axis β. The non-perpendicular direction can result in any non-zero angle θ between the cut distal end 128 and axis β. As shown in FIGS. 14A and 14B, the cut distal end 128 (which is also the distal end 108b of the shorter tube 104b) terminates proximal to the distal end 108a of the longer tube 104a by the length L. However, as shown in FIG. 16, also including a lumen tip segment 132 that has been attached to the cut distal end 128, the distal end 108b of the shorter tube 104b terminates the length L (less than a length L2 between the cut distal end 128 and the distal end 108a of the longer tube 104a) from the distal end 108a of the longer tube 104a. The lumen tip segment 132 can be made from a material different from a material of the shorter tube 104b (or whichever tube to which the lumen tip segment 132 is attached). The different material can be one more or less flexible than the material of the cut tube 104b. Using different materials for the lumen tip segment 132 and the cut tube 104b can allow the catheter assembly 100 to be used more efficiently or to be used at all in an application where it would not be preferable or possible having material of the cut tube 104b at the distal portion 114.

With a distal portion of the catheter assembly 100 removed, the lumen tip segment 132 can be joined to the catheter assembly 100, as shown in FIG. 16. The lumen tip segment 132 has been joined to the cut tube 104b at the cut distal end 128 such that the pathway of the cut tube 104b is in communication with the pathway of the lumen tip segment 132, thereby forming a single pathway 106b through the cut tube 104b and the lumen tip segment 132.

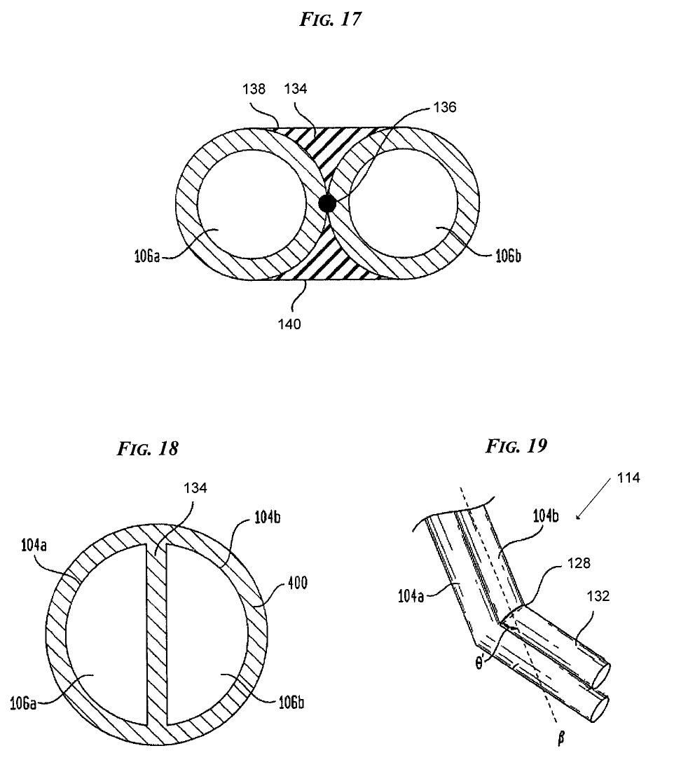

FIG. 16 also illustrates an embodiment of the catheter assembly 100 where the tubes 104 have been secured together along the facing surfaces 110 with a bioresorbable adhesive 134 for a length L5 between the cut distal end 128 and the distal end 108b of the shorter tube 104b. FIGS. 17-18 show cross-sections of the distal portion 114 of the tubes 104 detailing alternate embodiments of the bioresorbable adhesive 134 application. FIG. 17 shows the bioresorbable adhesive 134 applied at a contact point 136 of the facing surfaces of the tubes 104. FIG. 17 also shows one embodiment of an application of the bioresorbable adhesive 134 such that the adhesive 134, as applied, joins non-contacting surfaces 138, 140 of the tubes 104. FIG. 18 shows a variation on the embodiment shown in FIG. 17 where the bioresorbable adhesive 134 surrounds the tubes 104 forming a continuous cross-section of adhesive coating notwithstanding the pathways 106 extending therethrough. As stated above, the bioresorbable adhesive 134 need not be applied along the entire length of the tubes 104 but is preferably applied such that the adhesive 134 facilitates the joining of the distal extraction and return tip portions of the blood extraction and blood return tubes 104 prior to insertion into a blood vessel and allows the tubes 104 to separate after insertion. Furthermore, the tubes 104 can have different coatings from one another and/or different from a coating on the catheter assembly 100.

Referring again to FIG. 16, the lumen tip segment 132 can be attached to the catheter assembly 100 in a variety of ways. For example, the lumen tip segment 132 can be fused to the shorter tube 104b at the cut distal end 128. Any fusion technique can be used, e.g., thermal fusion where elements to be joined (here, the lumen tip segment 132 and the shorter tube 104b) are heated along any or all portions of their perimeters or other areas to a desired temperature and fused together by application of a desired force or by inserting one tube over the other (e.g., with an overlap by about 1 cm) and allowing them to melt/cool together. In another example, the lumen tip segment 132 can be bonded to the cut distal end 128. Any bonding technique can be used, e.g., applying a bonding material such as an adhesive to one or more of the elements to be bonded and, if necessary, heating the bonding material to bond it to the elements. In some embodiments, the lumen tip segment 132 can be attached in such a way as to provide a gradual transition between the luminal walls of the catheter assembly 100 and the luminal walls of the lumen tip segment 132, for instance via the insertion of a mandrel and the application of heat. The lumen tip segment 132 can also be formed from part of the longer tube 104a itself.

The lumen tip segment 132 can be oriented at any angle with respect to the longitudinal axis β of the cut tube 104b. Moreover, one or both of the lumen tip segment 132 and the distal tip portion 102a of the longer tube 104a (The tube to which the lumen tip segment 132 is not attached) can have a convex shape with respect to the other over at least some portion of its length. For example, the lumen tip segment 132 can be attached to the cut tube 104b at a ninety degree angle θ′ with respect to axis β, as shown in FIG. 16. In such a configuration, the distal portions 114 of the tubes 104 are separate but are substantially parallel to each other. FIG. 19 shows another embodiment where the distal portions 114 of the tubes 104 are separate and substantially parallel to each other in an angled spit tip configuration, e.g., as described in U.S. Pat. No. 6,482,169, which is hereby incorporated by reference in its entirety. Alternatively, as shown in FIG. 20, the lumen tip segment 132 can be oriented to the cut tube 104b at an angle θ′ less than ninety degrees. In such a configuration, the tubes 104 are separate and diverge from each other at an angle σ. When the angle θ′ is less than ninety degrees, it is typically in configurations where the cut distal end 128 has been trimmed in a non-perpendicular direction with respect to axis β, and the angle σ is formed when the lumen tip segment 132 is joined to the cut tube 104b. However, the angle σ can be formed after the lumen tip segment 132 has been joined to the cut tube 104b, e.g., by the application of heat. In another example, the design in FIG. 20 can be formed by first attaching the lumen tip segment 132 to the cut tube 104b and then heating the tube 104 to form the angle σ. Alternatively, the distal portion 114 such as that in FIG. 20 can have an initial configuration where the tubes 104 are at the angle θ′ with respect to axis β.

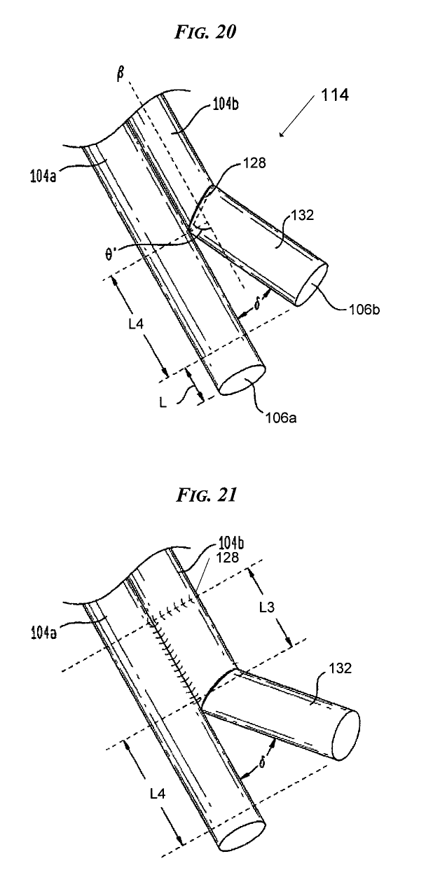

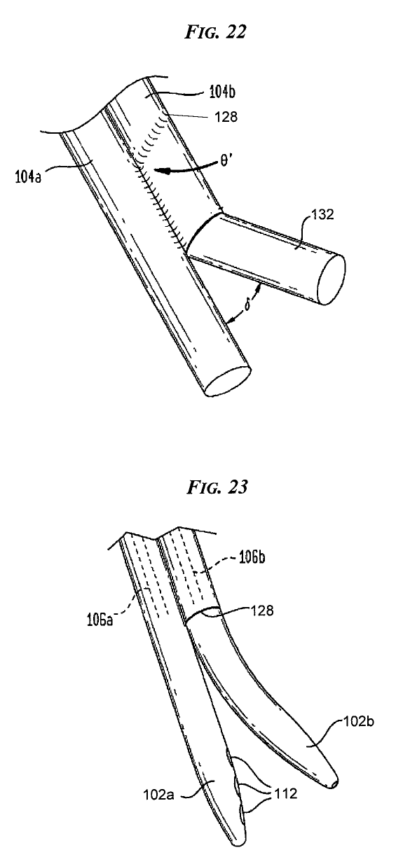

The apex of angle σ can be located either at the junction of the cut tube 104b and the lumen tip segment 132, as shown in FIG. 20, or further toward the distal ends 108 of the tubes 104. In the case that angle σ is further toward the distal end of the catheter assembly 100, the lumen tip segment 132 can be bonded to the septum along a length L3 of the uncut tube 104a, as shown in FIG. 21. Alternatively, the lumen tip segment 132 can be bonded to the septum along the length L3 of uncut tube 104a and attached to the cut tube 104b at a non-perpendicular angle θ′, as shown in FIG. 22. Typically, in these or other embodiments, the lumen tip segment 132 can also be bonded along the circumference at the junction with the cut tube 104b.

Whether substantially parallel or diverging from one another, the distal tip portion 102a and the lumen tip segment 132 are separate (at least before application of any adhesive, discussed further below). FIGS. 20-21 show the tubes 104 separate for the length L4.

FIG. 23 shows another embodiment where distal fluid openings 112 are formed in the distal tip portion 102a of the longer tube 104a. It should be understood from the drawings that in the embodiment shown, the distal fluid openings 112 can either be in addition to, or in place of, the pathway 106a opening located at the distal end 108a of the longer tube 104a. Furthermore, the shorter tube 104b can have distal fluid openings 112 similar to those described here, whereby the fluid openings 112 would typically be included in the distal tip portion 102b and, if the lumen tip segment 132 is present, be included in the lumen tip segment 132 or subsequently formed in the lumen tip segment 132 after its attachment to the catheter body 100.

Regardless of how the catheter assembly 100 is formed or whether a lumen tip segment is attached to one or both of the tubes 104, a flow diverting structure can be attached to the longer one of the tubes 104. Additional information on flow diverting structures and manufacturing techniques for catheters including flow diverting structures can be found in commonly-owned, co-pending U.S. patent application Ser. No. ______ filed concurrently herewith entitled “Manufacture of Fixed Tip Catheters” (Attorney Docket No. 101430-240).

FIG. 24 illustrates one embodiment of the catheter assembly 100 including a flow diverting structure 142. The diverting structure 142 can have any shape and size. The diverting structure 142 is shown having a width Wd that equals the width Wt of the tubes 104, but the diverting structure's width Wd can be greater than, less than, or equal to the width Wt of either or both the tubes 104. Similarly, the diverting structure 142 is shown having a diameter or height H3 that equals the diameter H2 of the shorter tube 104b, but the diverting structure's diameter H3 can be greater than, less than, or equal to the diameter of either or both the tubes 104. The diameter H3 of the diverting structure 142 can vary along a length Ld of the diverting structure 142 and/or along the width Wd of the diverting structure 142, e.g., if the diverting structure 142 has a non-perpendicular edge at either or both of its proximal and distal ends 144, 146, has a D-shaped cross-sectional shape (as shown in FIG. 24), includes one or more depressions and/or one or more protrusions anywhere on its surface, etc. Whether the diameter H3 of the diverting structure 142 varies or remains constant along the length Ld and/or the width Wd, a maximum value of the diameter H3 can be equal to or less than the diameter H2 of the shorter tube 104b, a configuration that can allow for easier insertion of the tubes 104 and the diverting structure 142 into the body when the diverting structure 142 has been attached to the longer tube 104a because the diverting structure 142 does not exceed the height H2 of the shorter tube 104b. As mentioned above, the diverting structure 142 as shown has a D-shaped cross-section having a constant area along the length Ld of the diverting structure 142, but the diverting structure 142 can have any cross-sectional shape, and its cross-sectional shape can change along its longitudinal length Ld. The diverting structure 142 can be solid or include one or more hollow cavities. Moreover, the diverting structure 142 can have a smooth outside surface, a textured outside surface, or a combination of both.

The flow diverting structure 142 has been attached to an outside surface of the longer tube 104a between the tubes' distal ends 108a, 108b, e.g., on the longer tube's facing surface 110a in the distal tip portion 102a. Examples of the diverting structure 142 are disclosed in Siegel, Jr. et al. U.S. Pat. No. 6,409,700. The diverting structure 142 can be attached anywhere on the longer tube 104a such that the diverting structure 142 is oriented to divert fluid flowing out of the pathway 106a at the distal end 108a of the longer tube 104a away from the pathway 106b at the distal end 108b of the shorter tube 104b. For example, the diverting structure 142 can be attached on the facing surface 110a of the longer tube 104a to intersect a longitudinal axis A of the shorter tube 104b. In this way, the diverting structure 142 can at least partially obscure a predicted path of fluid flowing into the distal end 108b of the shorter tube 104b. The diverting structure 142 is typically attached so its proximal end 144 is a distance D from the distal end 108b of the shorter tube 104b so as to provide adequate space for fluid to flow into the shorter tube 104b. The distance D can have any positive value less than the length L between the distal ends 108 of the tubes 104.

The diverting structure 142 can be attached to the longer tube 104a in a variety of ways. For example, in one embodiment, the diverting structure 142 can be fused to the longer tube 104a along at least a portion of a substantially flat surface (e.g., a facing or contacting surface 148) of the diverting structure 142 and along at least a portion of an outside surface of the longer tube 104a (e.g., on the facing surface 110a along a portion of the length L). Any fusion technique can be used, e.g., thermal fusion where elements to be joined (here, facing surfaces 110a, 148 of the longer tube 104a and the diverting structure 142, respectively) are heated along any or all portions of their perimeters or other areas to a desired temperature and fused together by application of a desired force and allowing them to melt/cool together. In another example embodiment, the diverting structure 142 and the longer tube 104a can be attached together using a gluing technique, e.g., applying a bonding material such as an adhesive to one or more of the elements to be bonded and, if necessary, heating the bonding material to bond it to the elements. In some embodiments, the catheter assembly 100 can be formed using any combination of heat fusion and gluing techniques.

The diverting structure 142 can be made of any biocompatible material which allows it to maintain structural integrity when in contact with flowing fluid, such as when inserted in a blood vessel during hemodialysis. The diverting structure's material can be the same as or different from that of the longer tube 104a and/or the shorter tube 104b. Using a different material for the diverting structure 142 (e.g., a harder material having a higher durometer) than for one or both of the tubes 104 can help create a more predictable fluid flow path by reducing chances of the diverting structure 142 flexing, bending, or otherwise distorting when being inserted into a fluid flow path (e.g., a blood vessel) and when fluid flows against the diverting structure 142.

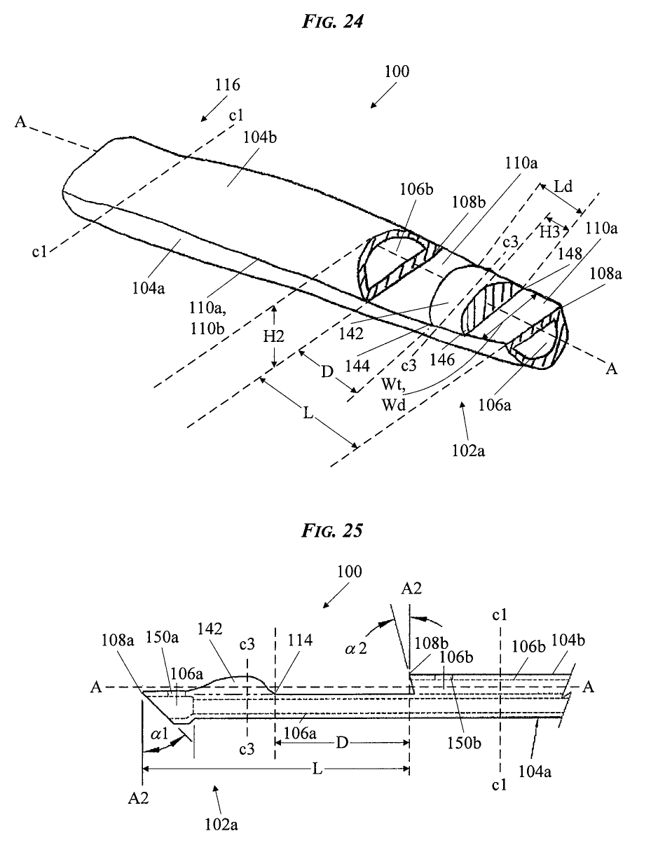

The distal ends 108 of the tubes 104 can each have any angle α1, α2 with respect to the transverse axes A2 of the tubes 104. The values of the angles α1, α2 can be the same or different. In the embodiment illustrated in FIG. 25, the longer tube 104a has an angle α1 equal to forty-five degrees, while the shorter tube 104a has an angle α2 equal to fifteen degrees. If the tubes 104 have beveled edges (e.g., if the angles α1, α2 are each above zero degrees but less than ninety degrees), the tubes 104 can be easier to insert into a body lumen.

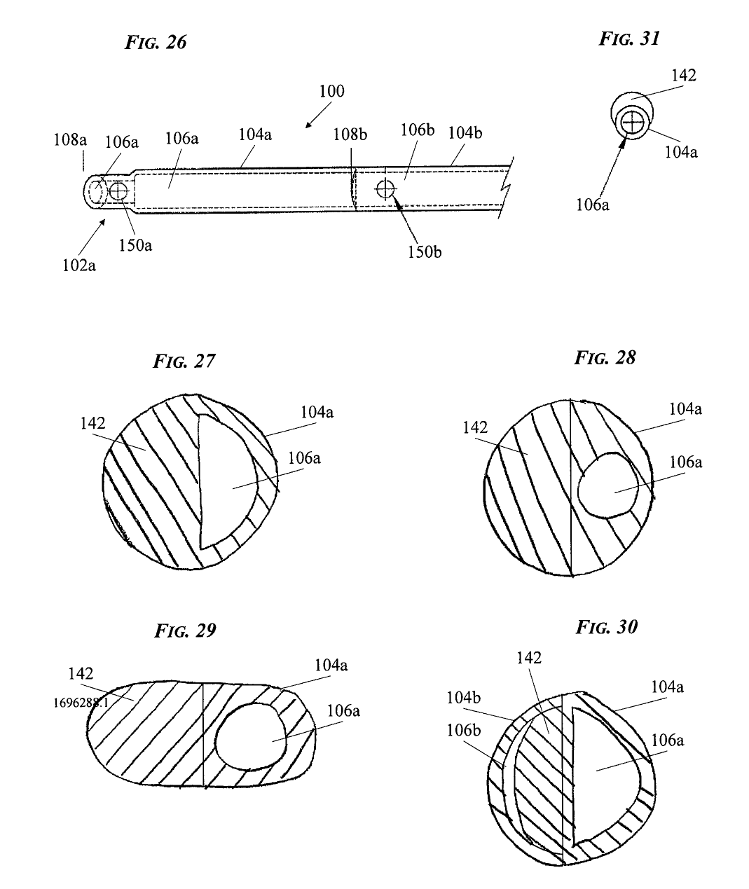

As shown in FIGS. 25 and 26, the first and second tubes 104a, 104b can optionally each include first and second holes 150a, 150b (collectively, the holes 150) in their respective surfaces and in communication with their respective pathways 106a, 106b. Although only one hole 150 is shown in each of the tubes 104, the tubes 104 can each include one or more holes 150 (if the tubes 104 include any at all). When the catheter assembly 100 is in use, the holes 150 can help relieve pressure and reduce clogging in the pathways 106. The holes 150 can also aid in inserting the catheter assembly 100 into a body lumen using a guidewire. A guidewire can be threaded into the first tube's pathway 106a at the distal end 108a, out of the first tube 104a through the first hole 150a, and into the second tube's pathway 106b through the second hole 150b. So threaded in the tubes 104, the catheter assembly 100 can be inserted over the guidewire into a body lumen.

FIG. 25 also illustrates a catheter assembly embodiment including a transition from one shape to another along at least a portion of a tube's length, e.g., transition from a D-shaped cross-section to a circular cross-section. In this example, the longer tube 104a has a D-shaped cross-section and a D-shaped pathway 106a except in at least part of the distal tip portion 102a, which has a circular-shaped cross-section and a circular-shaped pathway 106a. Having a circular-shaped distal portion with a rounded end, as shown, can allow for easier insertion of the longer tube 104b into a body lumen. The differently shaped part of the distal tip portion 102a can be a lumen tip segment that has been joined to the longer tube 104a, such as in the manner described above.

Examples of c3-c3 cross-sections (see FIGS. 24 and 25) including the diverting structure 142 and the longer tube 104a are illustrated in FIGS. 27-31. FIG. 27 shows a c3-c3 cross-section view of an embodiment showing a construction having a D-shaped tube 104a and a D-shaped diverting structure 142 having substantially the same cross-sectional area as the longer tube 104a. FIG. 28 is a c3-c3 cross-section view of an embodiment showing an elliptical construction utilizing a D-shaped diverting structure 142 and a D-shaped tube 104a having an individual, elliptical lumen pathway 106a. FIG. 29 is a c3-c3 cross-section view of another embodiment showing another elliptical construction including a D-shaped diverting structure 142 and an elliptical-shaped pathway 106a in the tubes 104a. FIG. 30 is a c3-c3 cross-section view of an embodiment showing a construction having D-shaped tubes 104 and a D-shaped diverting structure 142 having a smaller diameter and a smaller cross-sectional area than the shorter tube 104b. FIG. 31 shows another view of a c3-c3 cross-section of an embodiment showing a construction having a circular-shaped longer tube 104a and pathway 106a and a crescent-shaped diverting structure 142 curved around the outside surface of the longer tube 104a.

Although the examples of c3-c3 cross-sections in FIGS. 27-31 show the diverting structure 142 as solid, as mentioned above the diverting structure 142 can include one or more hollow portions. In such a case, the catheter assembly's c3-c3 cross section could be, for example, as shown in FIGS. 6-8. As also mentioned above, the diverting structure 142 can have a variable diameter (as measured between an outside surface of the diverting structure 142 to the contacting surface 110 of the tube 104 to which it is attached) along its longitudinal length Ld and/or width Wd, in which case its cross section can vary in size and/or shape along its longitudinal length Ld and/or width Wd.

Prior to the distal ends 108 of the catheter assembly 100 being inserted into a blood vessel, any or all portions of the distal tip portion 102 can be coated with at least one agent, such as an antithrombotic agent, an antibacterial agent, and an anti-inflammatory agent. By way of non-limiting example, antithrombotic agents such as heparin and hirudin, citrate, antithrombin-heparin complex, and albumin heparin complex as well as anti-infective agents such as chlorohexidine, silver, antibiotics, and antiseptic agents can be used. The agent can be applied along the distal tip portion 102 as a continuous coating or as a coating in discrete spots or regions. The spots or regions can vary in number, size, and distance from one another.

Other embodiments are within the scope of the following claims.

All publications, patent documents and other information sources identified in this application are hereby incorporated by reference.

What is claimed is:

1. A catheter assembly, comprising:
a first elongate catheter tube having a substantially D-shaped cross-section over at least a portion of its length and having a first lumen extending longitudinally through the first catheter tube; and
a second elongate catheter tube adjacent to the first catheter tube, extending a longitudinal length beyond a distal end of the first catheter tube, having a substantially D-shaped cross-section over at least a portion of its length, and having a second lumen extending longitudinally through the second catheter tube, the first lumen having a larger cross-sectional size than the second lumen over at least a portion of its length.

2. The assembly of claim 1, wherein at least one fluid passage hole is formed in a side of a distal portion of at least one of the catheter tubes.

3. The assembly of claim 1, wherein a hole is formed at a distal end of at least one of the catheter tubes.

4. The assembly of claim 1, wherein at least one of the catheter tubes includes a plurality of fluid passage holes.

5. The assembly of claim 1, wherein the assembly further comprises a lumen tip segment joined to a distal end of at least one of the catheter tubes such that the lumen tip segment is in communication with the lumen of the catheter tube to which it is joined.

6. The assembly of claim 1, wherein the first and second catheter tubes are a unibody construction with the first and second lumens separated by a longitudinal septum.

7. The assembly of claim 1, wherein the first and second catheter tubes are discrete elements and a longitudinal length of the first catheter tube is attached to a portion of a longitudinal length of the second catheter tube.

8. The assembly of claim 7, wherein the catheter tubes are attached together along at least about 70% of a longitudinal length of the second catheter tube.

9. The assembly of claim 7, wherein the catheter tubes are attached together along at least about 90% of a longitudinal length of the second catheter tube.

10. The assembly of claim 1, wherein proximal portions of the tubes are separate from each other.

11. The assembly of claim 1, wherein distal portions of the tubes are separate from each other.

12. The assembly of claim 1, wherein the assembly further comprises an outer sheath encasing the tubes along at least a portion of longitudinal lengths of the tubes.

13. The assembly of claim 1, wherein the assembly further comprises a flow diverting structure attached to an outside surface of a distal portion of the second catheter tube.

14. A catheter assembly, comprising:
two tubes disposed adjacent to each other along at least a portion of respective longitudinal lengths of the tubes, each tube having a lumen extending longitudinally therethrough, wherein a distal portion of one tube extends longitudinally beyond a distal portion of the other tube, and wherein one tube has a larger luminal cross-sectional size than the other tube over at least a portion of their lengths; and
a flow diverting structure attached to the distal portion of the tube that extends longitudinally beyond the other tube.

15. The assembly of claim 14, wherein the tubes are a unibody construction having a longitudinal septum extending therethrough.

16. The assembly of claim 14, wherein the tubes are attached together along at least about 70% of the longitudinal length of at least one of the tubes.

17. The assembly of claim 14, wherein the tubes are attached together along at least about 80% of the longitudinal length of at least one of the tubes.

18. The assembly of claim 14, wherein the tubes are attached together along at least about 90% of the longitudinal length of at least one of the tubes.

19. The assembly of claim 14, wherein the tubes each have at least one flat surface and the tubes are attached together along their flat surfaces.

20. The assembly of claim 14, wherein the tubes each have substantially D-shaped cross-sections.

21. The assembly of claim 14, wherein one of the first and second tubes has a cross-section shape different from the other tube along at least a portion of its longitudinal length.

22. The assembly of claim 14, wherein proximal portions of the tubes are separate from each other.

23. The assembly of claim 14, wherein at least one fluid passage hole is formed in a side of a distal portion of at least one of the tubes.

24. The assembly of claim 14, wherein a hole is formed at a distal end of at least one of the tubes.

25. The assembly of claim 14, wherein at least one of the tubes includes a plurality of fluid passage holes.

26. The assembly of claim 14, wherein the assembly further comprises an outer sheath encasing the tubes along at least a portion of their attached longitudinal lengths.

27. The assembly of claim 14, wherein the flow diverting structure is oriented to intersect a longitudinal axis of the tube to which it is not attached.

28. The assembly of claim 14, wherein the flow diverting structure is composed of a material different than a material of the tube to which it is attached.

29. The assembly of claim 28, wherein the flow diverting structure is composed of a material with a durometer that differs from that of the tube to which it is attached.

30. The assembly of claim 14, wherein a diameter of the flow diverting structure does not exceed a diameter of the tube to which it is not attached.

31. The assembly of claim 14, wherein the flow diverting structure is fused to the distal portion of the tube that extends longitudinally beyond the other tube.

32. The assembly of claim 14, wherein the flow diverting structure is glued to the distal portion of the tube that extends longitudinally beyond the other tube.

33. The assembly of claim 14, wherein the flow diverting structure is attached at a location between distal ends of the tubes.

34. A catheter assembly, comprising:
a first catheter tube having a first inner lumen extending longitudinally therethrough; and
a second catheter tube having a length greater than a length of the first catheter tube and having a second inner lumen extending longitudinally therethrough, the first inner lumen having a cross-sectional size that is larger than a cross-sectional size of the second inner lumen, and wherein a distal portion of the first inner lumen has a cross-sectional size that is different from the cross-sectional size in its non-distal portion.

35. The assembly of claim 34, wherein the first and second catheter tubes each have substantially D-shaped cross-sections.

36. The assembly of claim 34, wherein the cross-sectional size of the distal portion of the first inner lumen is larger than the cross-sectional size in its non-distal portion.

37. The assembly of claim 34, wherein at least one fluid passage hole is formed in a side of a distal portion of at least one of the catheter tubes.

38. The assembly of claim 34, wherein a hole is formed at a distal end of at least one of the catheter tubes.

39. The assembly of claim 34, wherein at least one of the catheter tubes includes a plurality of fluid passage holes.

40. The assembly of claim 34, further comprising a lumen tip segment joined to a distal end of the first catheter tube such that the lumen tip segment is in communication with the first inner lumen.

41. The assembly of claim 34, wherein the first and second catheter tubes are a unibody construction with the first and second inner lumens separated by a longitudinal septum.

42. The assembly of claim 34, wherein the first and second catheter tubes are discrete elements and a longitudinal length of the first catheter tube is attached to a portion of a longitudinal length of the second catheter tube.

43. The assembly of claim 34, wherein proximal portions of the tubes are separate from each other.

44. The assembly of claim 34, wherein distal portions of the tubes are separate from each other.

45. The assembly of claim 34, wherein one of the first and second inner lumens has a cross-sectional shape different from the other inner lumen along at least a portion of its longitudinal length.

46. The assembly of claim 34, wherein the assembly further comprises an outer sheath encasing the first and second catheter tubes along at least a portion of longitudinal lengths of the tubes.

47. The assembly of claim 34, wherein the assembly further comprises a flow diverting structure attached to a distal portion of the second catheter tube.
